(19) United States
(12) Patent Application Publication
(10) Pub. No.: US 2010/0121502 A1
(43) Pub. Date: May 13, 2010

(54) Operation control system and method

(75) Inventors: Osamu Katayama, Nagoya-city (JP); Keisuke Hachisuka, Nagoya-city (JP); Ryoichi Sugawara, Nagoya-city (JP)

Correspondence Address: POSZ LAW GROUP, PLC, 12040 SOUTH LAKES DRIVE, SUITE 101, RESTON, VA, 20191, US

(73) Assignee: DENSO CORPORATION, Kariya-city, Aichi-pref (JP)

(21) Appl. No.: 12/591,058

(22) Filed: Nov. 5, 2009

(30) Foreign Application Priority Data
Nov. 7, 2008 (JP) 2008-286475

(51) Int. Cl. G06F 7/00; A61B 5/00; G06F 7/04

(52) U.S. Cl. 701/1; 600/309; 600/532; 340/5.1

(57) ABSTRACT

An operation control system includes a transmitter circuit, a transmitter interface, an engine start button switch, a receiver unit and an engine ECU. The transmitter circuit generates a transmission signal and the transmitter interface inputs a communication signal to an operator by converting the transmission signal. The receiver unit receives the communication signal through the operator, when the operator touches the engine start button switch. The engine start button switch detects in-blood alcohol concentration of the operator. The receiver unit generates and applies an engine start control signal in response to output signals of the receiver unit indicative of the operator's engine start operation and alcohol detection result.

### CROSS REFERENCE TO RELATED APPLICATION

This application is based on and incorporates herein by reference Japanese Patent Application No. 2008-286475 filed on Nov. 7, 2008.

### FIELD OF THE INVENTION

The present invention relates to an operation control system and method, which controls operation of a control subject by inhibiting or permitting the operation of the control subject based on a check result of a human body condition of an operator.

### BACKGROUND OF THE INVENTION

It is conventional to check condition of a human body of an operator seated on an operator's seat of a device (control subject) before the device is operated by the operator, so that the operation is permitted or inhibited if the check result indicates that the body condition of the operator is suitable and not suitable for operation of the device, respectively.

For example, concentration of alcohol in driver's blood (in-blood alcohol concentration) is measured by checking breath air of a driver. If the measured in-blood alcohol concentration exceeds a predetermined threshold level, a drive power source (for example, an internal combustion engine or electric motor) of a vehicle is made inoperative thereby to disable drunk driving.

Such a technology is proposed in a few articles provided in the following Internet site as of Sep. 30, 2008.

URL:http://www.drivingfuture.com/car/volvo/2008/tech/080410#alcoguard/001.html

This article introduces an alcohol ignition interlock, which combines an ignition key with an alcohol detection device. If alcohol is detected in breath air blown out by, a driver before start of driving of a vehicle, an engine is prohibited from being started. In Sweden, it is proposed to detect the alcohol concentration in the driver's breath air blown out to a part of an engine key or a buckle part of a seatbelt. It is also under research that alcohol concentration is detected from skin of hands of a driver grasping a steering wheel.

URL: http://www.yomiuri.co.jp/atcars/news/20060923ve01.html

This article introduces “Alcoguard,” which is an alcohol interlock device. This device measures alcohol concentration in blood of a driver of a vehicle based on breath, air blown out by the driver to a vehicle-mounted handset. If the measured alcohol concentration is high, engine is prohibited from being started.

However, if a person, who is not drunk, takes such checking in place of a drunk driver seated on a driver's seat, the drive power source may be operated by the drunk driver.

The control subject subjected to the inhibition of operation is not limited to vehicles such as cars and airplanes, but may be computers, power plant control devices, which are, operated by operators seated on operators' seats.

Measurement is not limited to the in-blood alcohol concentration. Blood sugar level may also be measured, because high blood sugar level is likely to lower sense of responsibility and judgment required for operating various devices as control subjects.

### SUMMARY OF THE INVENTION

It is therefore an object of the present invention' to provide an operation control system and method, which disables change of setting of an inhibition state of a control subject even if a person other than an actual operator seated on an operator's seat takes a required check of body condition.

According to one aspect of the present invention, an operation control system for a control subject includes a setting section, a communication section, and a condition checking section. The setting section is configured to set the control subject to an inhibition state or a permission state based on a body condition of an operator seated on a seat for operating the control subject, so that the operation of the control subject is limited in the inhibition state and permitted in the permission state. The communication section includes a transmitter and a receiver and is configured to perform communication through a human body of the operator, which is present closely to the transmitter and the receiver. The condition checking section includes a detector and is configured to check the body condition of the operator under a condition that the detector is positioned closely to the operator. One of the transmitter and the receiver is provided at the seat and the other of the transmitter and the receiver is provided at the detector of the condition checking section. The setting section is configured to set the inhibition state or the permission state based on a check result of the condition checking section outputted in a condition that the human body communication is established.

### BRIEF DESCRIPTION OF THE DRAWINGS

The above and other objects, features and advantages of the present invention will become more apparent from the following detailed description made with reference to the accompanying drawings. In the drawings:

FIG. 1 is a schematic diagram of an operation control system according to the first embodiment of the present invention;

FIG. 2 is a block diagram of the operation control system according to the first embodiment;

FIG. 3 is a flowchart of an engine start control routine executed in the first embodiment;

FIG. 4 is a flowchart of a driver change monitoring routine executed in the first embodiment;

FIG. 5 is a schematic diagram of an operation control system according to the second embodiment of the present invention;

FIG. 6 is a block diagram of the operation control system according to the second embodiment;

FIG. 7 is a flowchart of an engine start control routine executed in the second embodiment;

FIG. 8 is a schematic diagram of an operation control system according to the third embodiment of the present invention;

FIG. 9 is a block diagram of the operation control system according to the third embodiment;

FIG. 10 is a table showing a setting of a personal computer relative to conditions of a human body communication, an alcohol concentration and a control signal in the third embodiment;

FIG. 11 is a flowchart of a log-in control routine executed in the third embodiment;

FIG. 12 is a flowchart of an operator change monitoring routine executed in the third embodiment;

FIG. 13 is a schematic diagram of an operation control system according to the fourth embodiment of the present invention;

FIG. 14 is a block diagram of the operation control system according to the fourth embodiment;

FIG. 15 is a flowchart of a personal computer operation control routine executed in the fourth embodiment; and

FIG. 16 is a flowchart of a partial control routine executed in the operation control routine shown in FIG. 15.

### DETAILED DESCRIPTION OF THE PREFERRED EMBODIMENT

The present invention will be described in detail with reference to embodiments shown in the drawings, in which the same or similar parts are denoted by the same or similar reference numerals so that the same or similar description may be simplified.

### First Embodiment

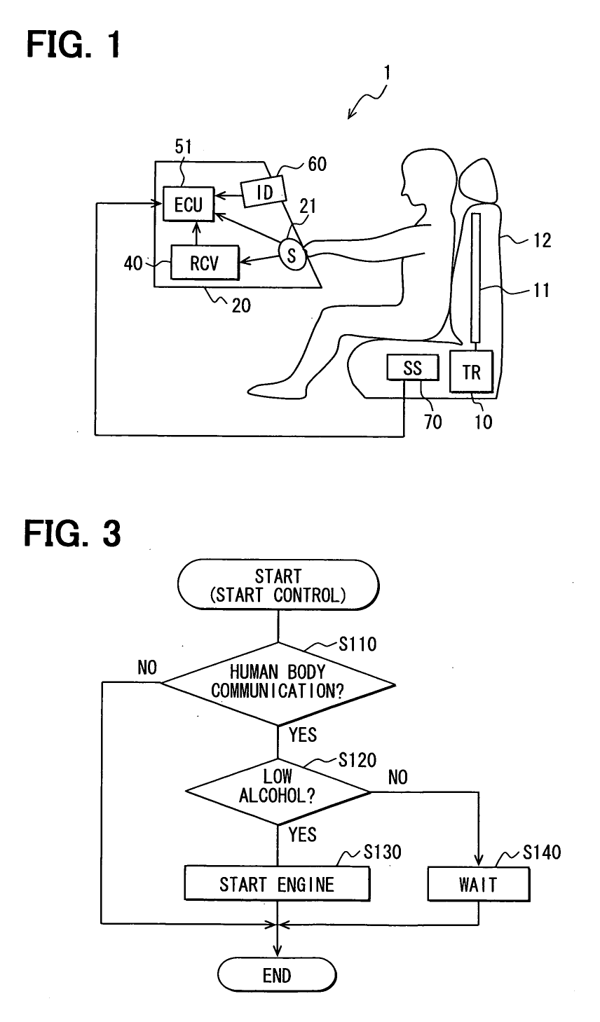

Referring to FIG. 1, an operation control system 1 is mounted on a vehicle for preventing drunk driving. The vehicle is a control subject to be controlled. In this system, alcohol concentration in blood (in-blood alcohol concentration) of a person seated as a driver on a driver's seat is measured as a body condition of an operator before starting a vehicle engine, which is a part of the control subject. If the measured in-blood alcohol concentration exceeds a predetermined threshold level, the engine is disabled from being started. If the engine is operated by a person other than the person, who passed the in-blood alcohol concentration measurement, after the engine has been started, the engine is stopped from being further operated thereby to surely eliminate possibility of drunk driving.

The operation control system is configured with a transmitter circuit 10, a transmission interface (I/F) 11, an engine start button switch (S) 21, a receiver unit (RCV) 40 an engine electronic control unit (ECU) 51, an image detector unit (ID) 60 and a seat sensor (SS) 70.

The transmitter circuit 10, the transmitter interface 11, and the seat sensor 70 are provided in or on an operator's seat 12, which is a driver's seat in a vehicle. The engine start button switch 21, the receiver unit 40, the engine ECU 51 and the image sensor 60 are provided in or on an instrument panel 20 of the vehicle, which faces the operator's seat 12.

The transmitter circuit 10 is configured to generate a transmission signal for human body communication. The transmitter interface 11 is provided at the backrest of the seat 12 to convert the generated transmission signal into a communication signal and transmit the transmission signal toward the back part of a driver seated on the operator's seat 12. The transmission signal is transmitted to the engine start button switch 21 through the human body of the driver, when the driver operates the engine start button switch 21 to start an engine (not shown). The receiver unit 40 is connected to the engine start button switch 21 to receive the transmission signal as a reception signal. The engine ECU 51 is connected to the engine start button switch 21 to receive an engine operation signal, which is generated when the engine start button switch 21 is operated to start the engine.

The engine start button switch 21 is configured to measure the in-blood alcohol concentration of the driver and transmit a check signal corresponding to the measured in-blood alcohol concentration to the receiver unit 40, when the driver finger-touches the engine start button switch 21. The receiver unit 40 is configured to generate a control signal based on the reception signal and the check signal and transmit it to the engine ECU 51.

The image detector unit 60 is configured to take a picture around the operator's seat 12 by an image sensor for example, checks whether any person is seated on the operator's seat 12 by face recognition, whether a person seated on the operator's seat 12 has changed by recognized face comparison, and transmit information indicative of the check results to the engine ECU 51. The seat sensor 70 may be a pressure sensor provided at the bottom part of the operator's seat 12 and is configured to transmit information indicative of whether any person is seated on the seat 12 to the engine ECU 51.

The engine ECU 51 is configured to control the engine start operation based on the signals and information received, from the engine start button switch 21, the receiver unit 40, the image detector unit 60 and the seat sensor 70.

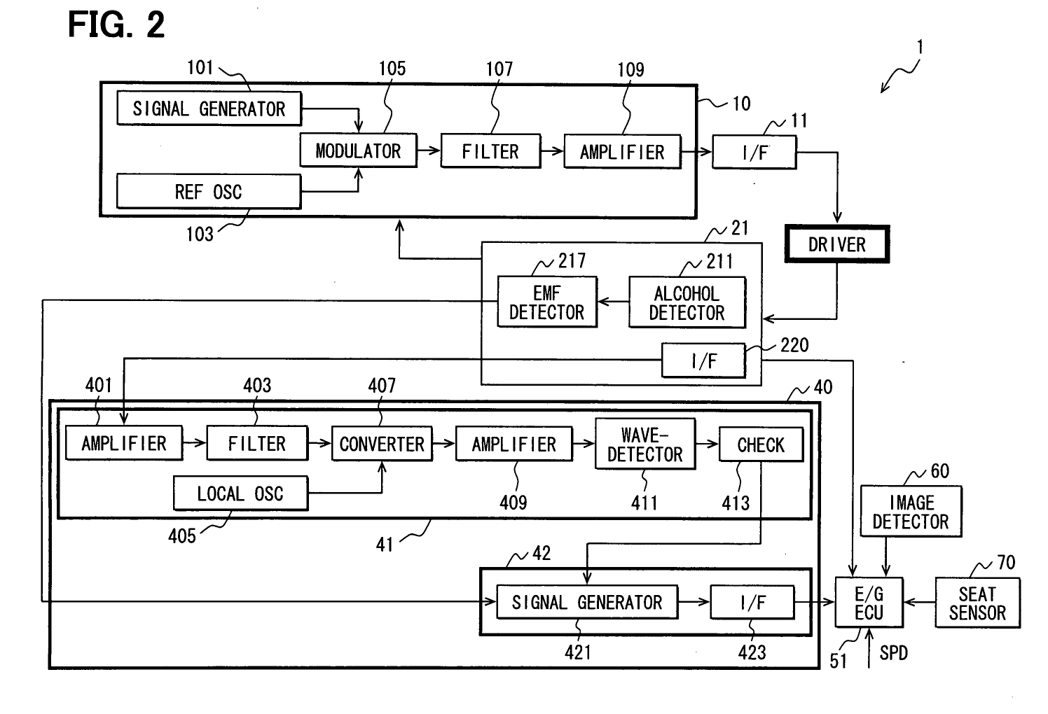

The electric configuration of the operation control system 1, is shown in FIG. 2 in more detail under an assumption that the driver is touching the engine start button switch 21 to start the engine. The transmitter circuit 10 includes a signal generator circuit 101, a reference oscillator circuit 103, a modulator circuit 105, a filter circuit 107 and an amplifier circuit 109.

The signal generator circuit 101 is configured to generate an original signal as a base of the transmission signal. The reference oscillator circuit 103 is configured to generate a reference signal as a carrier wave, which is modulated by the original signal.

The modulation circuit 105 is configured to generate a modulation signal by modulating a frequency or amplitude of the reference signal inputted from the reference oscillator circuit 103 by the original signal inputted from the signal, generator circuit 101. The modulation signal of the modulator circuit 105 is inputted to the filter circuit 107.

The filter circuit 107 is configured as a band-pass filter to attenuate frequency components (noise components) of a predetermined range included in the modulation signal inputted from the modulator circuit 105.

The modulation signal, in which noise components are attenuated by the filter circuit 107, is inputted to the amplifier circuit 109. The amplifier circuit 109 is configured to amplify the modulation signal received from the filter circuit 107 and transmit it to the transmitter interface 11.

The transmitter interface 11 is configured to convert the modulation signal received from the amplifier circuit 109 to a communication signal, which is inputted to the driver. The transmitter interface 11 is made up of a coil, which converts the modulation signal into magnetic field generated around the body of the driver. This magnetic field propagates to the driver, resulting in input of the modulation signal to the driver.

The transmitter circuit 10 is configured to be operable as long as the engine start button switch 21 is turned on, that is, as long as a start signal is applied from the engine start button switch 21, to the transmitter circuit 10. The frequency of the modulation signal, the output strength of the magnetic field, position of the transmitter interface 11 and the like are so determined that the magnetic field may propagate to the surface of the body of the driver seated on the operator's seat 12, in which the transmitter interface 11 is embedded.

The engine start button switch 21 includes a receiver interface 220 provided at the lower or bottom part, toward which the engine start button switch 21 is pressed down. The receiver interface 220 is configured to receive the magnetic field propagated to the driver and converts it into a reception signal, which is inputted to the receiver unit 40, as long as the driver is in touch with the engine start button switch 21.

The receiver unit 40 includes a receiver circuit 41 and a control signal generator circuit 42. The receiver circuit 41 includes an amplifier circuit 401, a filter circuit 403, a local oscillator 405, a frequency converter circuit 407, an amplifier circuit 409, a wave-detector circuit 411 and a check circuit 413. The control signal generator 42 includes a signal generator circuit 421 and a signal interface 423.

The reception signal inputted from the receiver interface 220 of the engine start button switch 21 is inputted to the amplifier circuit 401. The amplifier circuit 401 is configured to amplify the reception signal and inputs it to the filter circuit 403. The filter circuit 403 is configured as a band-pass filter to attenuate frequency components (noise components) of a predetermined range included in the reception signal inputted from the amplifier circuit 401. The reception signal, in which noise components are attenuated, is inputted to the frequency converter circuit 407.

The local oscillator circuit 405 is configured to generate a local oscillation signal and inputs it to the frequency converter circuit 407. The frequency converter circuit 407 subtracts the reception signal inputted from the filter circuit 403 from the local oscillation signal inputted from the local oscillator circuit 405. Thus, the reception signal is converted into an intermediate frequency signal, which is inputted to the amplifier circuit 409. The amplifier circuit 409 is configured to amplify the intermediate frequency signal and inputs the amplified intermediate frequency signal to the wave-detector circuit 411.

The wave-detector 411 is configured to demodulate the intermediate frequency signal inputted from the amplifier circuit 409 and input the demodulated signal to the check circuit 413. The check circuit 413 is configured to check whether the intermediate frequency signal is wave-detected properly, and inputs the wave-detected signal to the control signal generator 42.

The engine start button switch 21 further includes an optical alcohol detector 211 and an electromotive force detector 217. The optical alcohol detector 211 may be in a conventional configuration (for example, JP 2008-086724), in which the alcohol concentration in blood is detected by irradiating light onto a finger and sensing transmitted light. The detector 211 is configured to detect the in-blood alcohol concentration when the driver touches the engine start button switch 21 and generate an electromotive force corresponding to the measured in-blood alcohol concentration. The electromotive force detector circuit 217 is configured to detect the electromotive force and inputs a detection signal indicative of the detection result to the signal generator circuit 421.

The signal generator circuit 421 is configured to generate a control signal, which includes information of the detection signal inputted from the electromotive force detector circuit 217, when the check signal is inputted from the check circuit 413. The signal interface 423 is configured to transmit the control signal to the engine ECU 51.

The strength of the communication signal is set theoretically and experimentally so that the communication may be established through only one person as a propagation medium.

The engine start operation is controlled as a part of vehicle travel control based on engine start control routine shown in FIG. 3. This routine is performed primarily by the engine ECU 51 when the engine start button switch 21 is operated by being pressed down. This routine is performed with priority over other routines. That is; until this routine is completed after having been started once, no other routines are started even if the engine start button switch 21 is pressed down.

First, it is checked whether human body communication is established (S110). Specifically, it is checked whether the control signal is received through the interface 423. If it is determined that no human body communication is established (NO at S110), this control routine is finished. As a result, the engine is not started even if the engine start button switch 21 is operated.

If it is determined that the human body communication is established (YES at S110), it is further checked whether the measured in-blood alcohol concentration is lower than a predetermined threshold level (S120). Specifically, it is checked whether the information of the electromotive force indicative of the in-blood alcohol concentration measurement result and included in the control signal indicates low alcohol concentration. If it is determined that the in-blood alcohol concentration is low (YES at S120), the engine is started (S130) and this control routine is finished. The engine is thus set to a start permission state by releasing the engine from the inhibition state.

If it is determined that the in-blood alcohol concentration is higher than the predetermined threshold level (NO at S120), this control routine is finished after waiting for a predetermined wait period (S140). This predetermined wait period is provided for inhibiting starting of the engine. Thus, the engine is set to a start inhibition state and the engine is inhibited from starting. Even if the engine start button switch 21 is operated again to start the engine, the engine starting is not performed during this wait period because the processing S130 is not executed.

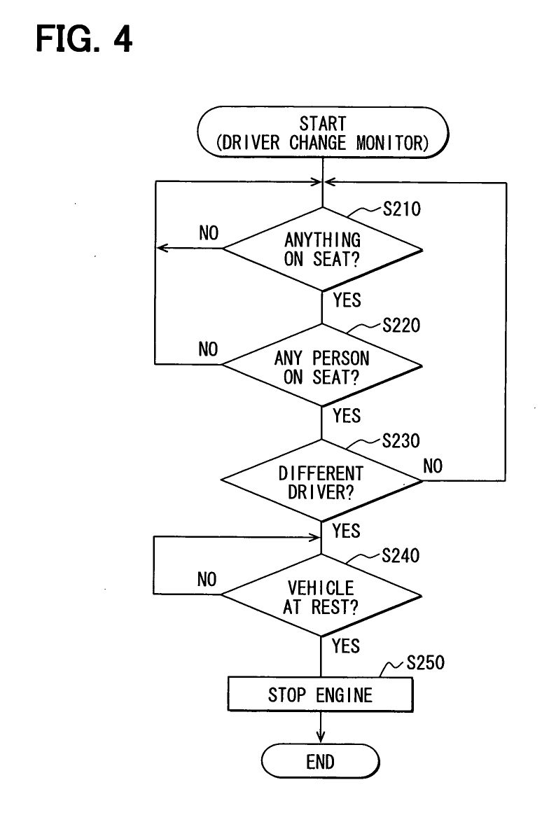

Any change of a driver is monitored by a driver change monitoring routine shown in FIG. 4. This routine is also primarily performed by the engine ECU 51 when the engine is started, for example, after the engine has been started.

It is first checked whether any thing or person is on the operator's seat 12 based on the information produced by the seat sensor 70 (S210). If it is determined based on the information of the seat sensor 70 that a certain thing or a person is on the operator's seat 12 (YES at S210), it is further checked based on information produced by the image detector unit 60 whether any person is seated on the operator's seat (S220).

If it is determined that nobody is on the operator's seat 12 based on the information of the seat sensor 70 and the image detector unit 60 (NO at both S210 and S220), the processing of S210 and S220 is repeated.

If it is determined that a person is seated on the operator's seat 12 (YES at S220), it is further checked whether a new person different from the last person, who was seated a moment ago, is seated on the operator's seat 12 (S230). Specifically it is checked whether the new person is different from the last person by comparing the image of the new person taken by the image sensor of the image detector unit 60 with the image of the last person taken by the image sensor and stored in the image detector unit 60. Thus, it is checked whether a driver has changed. If it is determined that the driver is the same and not different from the last person (NO at S230), the processing S210, S220 and S230 are repeated.

If it is determined that the driver is changed (YES at S230), it is checked whether the vehicle is at rest, that is, whether the travel speed of the vehicle is zero based on speed information provided through the in-vehicle local area network (S240). If it is determined that the vehicle is not at rest (NO at S240), this processing is repeated until the vehicle travel speed becomes zero. If it is determined that the vehicle is at rest (YES at S240), the engine is stopped (S250) thus finishing this driver change monitoring routine.

According to the first embodiment, drunk driving is prevented, because the engine is inhibited from being started by a drunk driver. Further, even when the engine is started by a driver, who is not drunk, the engine is automatically stopped from continuing to operate if the driver changes to a different person. Thus, the engine is set to the inhibition state again.

It often occurs that the driver changes in the course of travel of the vehicle. In this instance, since the engine is stopped after the vehicle comes to rest, it is prevented that the engine is stopped suddenly in the middle of traveling.

The signal strength of the communication signal is set to correspond to the human body communication of only one person, it is prevented that a person different from a drunk person and not drunk touches the engine start button switch 21 to start the engine by holding a hand of a drunk person, who is seated on the driver's seat.

The engine starting may be prevented in different ways, even when the human body communication is established by holding the hand of the drunk driver. In case a person holding the hand of the drunk driver seated on the driver's seat touches the engine start button switch 21 from the outside of the vehicle, the engine starting may be enabled only under a condition that a door or window next to the driver's seat is closed.

In case a person seated on a front passenger's seat next to the driver's seat holds a hand of the drunk driver on the driver's seat, a transmitter circuit and a transmitter interface may similarly be provided in the passenger's seat so that the magnetic fields generated at the operator's seat 12 and the passenger's seat cancel or counteract each other. It is also possible to generate a specific signal by the transmitter of the passenger's seat and disable the engine starting when the engine ECU 51 receives this specific signal.

The engine may be stopped automatically after being started, when a driver once leaves the driver's seat. Although it is troublesome that the driver must repeat the same starting operation again when returning to the vehicle, drunk driving due to erroneous verification of a driver caused in image processing can be prevented.

### Second Embodiment

An operation control system 2 according to the second embodiment is provided in a vehicle for disabling drunk driving by setting the control subject to the inhibition state, for example, by inhibiting the engine starting or forcibly stopping the engine operation when the measured in-blood alcohol concentration is more than a predetermined threshold level.

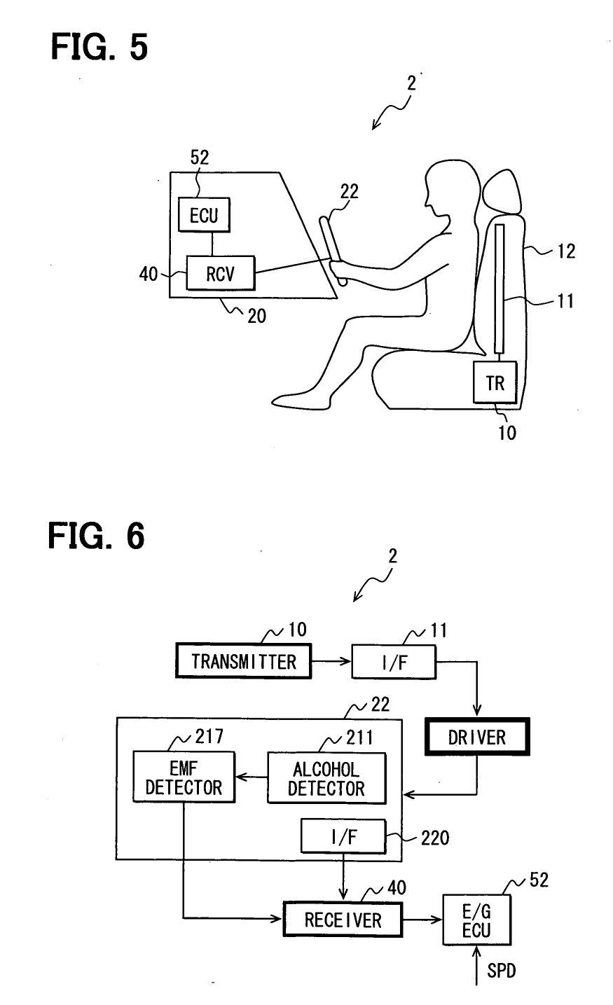

As shown in FIG. 5, the operation control system 2 includes an engine ECU 52 in addition to the transmitter circuit 10, the transmitter interface 11 and the receiver unit 40.

As in the first embodiment, the transmitter circuit 10 and the transmitter interface 11 are provided in the operator's seat 12 and the receiver unit 40 and the engine ECU 52 are provided in the instrument panel 20. A steering wheel 22 is provided outside the instrument panel 20 in the conventional manner.

The optical alcohol detector 211, the electromotive force detector circuit 217 and the receiver interface 220 are provided in or on the steering wheel 22. With this arrangement, when a driver holds the steering wheel 22, the in-blood alcohol concentration is measured by the alcohol detector 211 and hands of the driver seated on the operator's seat 12 are positioned closely to the receiver interface 220 so that the human body communication may be established.

The optical alcohol detector 211 is provided to extend circularly on the steering wheel 22 so that it may be touched by the driver's hands. If the steering wheel 22 is not a circular shape, the area or length of the alcohol detector 211 may be limited to be smaller or shorter in correspondence to the actual shape of the steering wheel, as long as it is touched by the driver.

The engine ECU 52 is configured to control the engine based on a control signal transmitted from the receiver unit 40.

The electric configuration of the operation control system 2 is shown in FIG. 6 in more detail under an assumption that the driver is seated on the operator's seat 12 and holding or touching the steering wheel 22. The transmission signal outputted from the transmitter circuit 10 is inputted to the driver as the communication signal through the transmitter interface 11. The communication signal is inputted to the receiver unit 40 as the reception signal through the driver and the receiver interface 220.

The electromotive force generated by the optical alcohol detector 211 is detected by the electromotive force detector circuit 217. The information of the detected electromotive force is inputted to the receiver unit 40 as the detection signal.

The receiver unit 40 generates the control signal based on the reception signal and the detection signal. The control signal is inputted to the engine ECU 52.

The transmitter circuit 10 is configured to operate when the engine start button switch (not shown) is operated by being pressed down under a condition that the engine is not in operation yet. It is configured to operate persistently as long as the engine is in operation.

The ECU 52 is configured to perform an engine start control routine in response to the control signal.

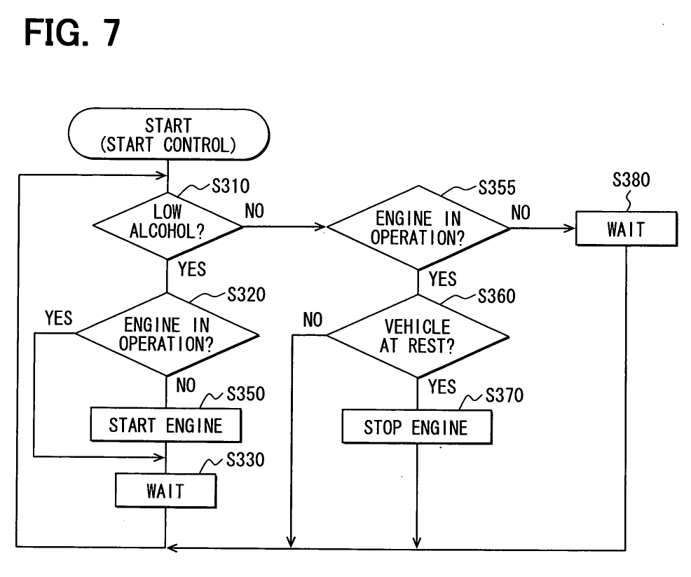

This routine is primarily performed by the engine ECU 52 as shown in FIG. 7, when the human body communication is established and forcibly finished when the human body communication is not established any more.

In the engine start control routine shown in FIG. 7, it is first checked whether the measured in-blood alcohol concentration is lower than a predetermined threshold level based on the control signal (S310). If it is determined that the alcohol concentration is low (YES at S310), the engine is set to the permission state. It is further checked whether the engine is in operation (S320). If it is determined that the engine is in operation (YES at S320), the processing returns to S310 after waiting for a predetermined wait period (S330). This wait period is for lengthening the processing interval so that this routine is not performed so frequently.

If it is determined that the engine is at rest and not in operation (NO at

S320), the engine is started (S350). After waiting for the predetermined wait period (S330), the processing returns to S310. Thus, the engine is started at S350, because it is only when the engine start button switch 21 is operated that this processing is started under the condition that the engine is at rest.

If it is determined that the alcohol concentration is high (NO at S310), it is checked whether the engine is in operation (S355). If the engine is not in operation (NO at S355), the processing returns to 5310 after waiting for a predetermined wait period (S380).

This wait period is for inhibiting the engine starting operation during the period. It is required to execute S350 to start the engine. However, if the processing proceeds to S355, S350 cannot be executed unless the processing is started again from S310. Therefore, during this wait period, the engine starting operation is inhibited. For this purpose, even if the human communication is established after it has not been established once, the next processing is not started immediately but is started only after such a wait period.

If it is determined that the engine is in operation (YES at S355), it is further checked whether the vehicle is at rest by checking whether the travel speed of the vehicle is zero based on the information acquired from the in-vehicle local area network (S360). If it is determined that the vehicle is not at rest (NO at S360), the processing returns to S310 and S360 is repeated. If it is determined the vehicle is at rest (YES at S360), the engine is stopped (S370) and the processing returns to S310. The engine is not stopped until the vehicle comes to at rest, because it is not proper to stop the engine while the vehicle is traveling.

According to the second embodiment, drunk driving is prevented. In particular, since the in-blood alcohol concentration is detected as long as the driver holds the steering wheel 22, the drunk driving can be prevented by stopping the engine even when the driver starts drinking after the engine is started. It is not necessary to monitor any change of the drivers, and hence hardware and software configuration are simplified. Since it is only the driver that establishes the human body communication and holds the steering wheel 22, it is prevented that the engine is started by checking any different persons other than the actual driver.

### Third Embodiment

An operation' control system 3 according to the third embodiment is provided for disabling a drunk person from operating a personal computer by inhibiting the log-in operation of the personal computer when the measured in-blood alcohol concentration of an operator is more than a predetermined threshold level. It is also inhibited that the personal computer is operated by a drunk person in case that the operator is changed from a normal person, who is not drunk, to the drunk person after completing the log-in of the computer by the normal person.

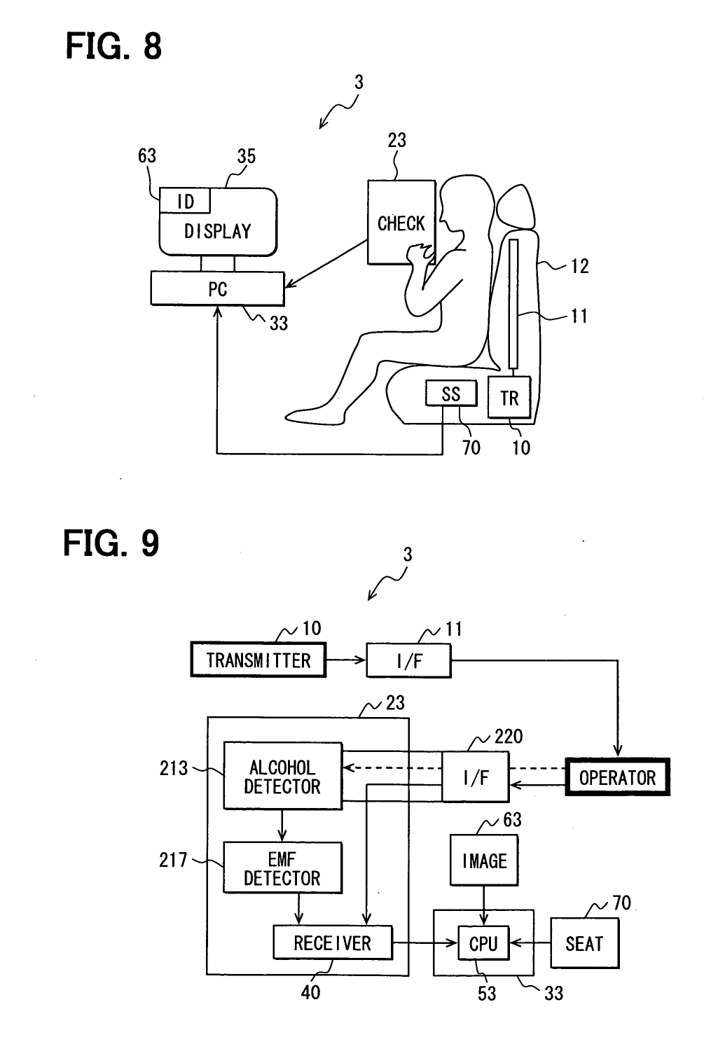

As shown in FIG. 8, the operation control system 3 includes a check unit 23, a personal computer (PC) 33, a display unit 35, an image detector unit 63 in addition to the transmitter circuit 10, the transmitter interface 11 and the seat switch 70, which are provided in the operator's seat 12.

The image detector unit 63 is provided at the corner of the display unit 63.

The check unit 23 is configured as an alcohol detector unit to measure the in-blood alcohol concentration by analyzing the breath of an operator. The check unit 23 is so configured to perform human body communication with the transmitter circuit 10 through the human body of the operator seated on the operator's seat 12 when the communication signal of the transmitter circuit 10 is applied to the operator. The check unit 23 is connectable to the personal computer 33 through a universal serial bus (USB: registered trademark). That is, it need not be integrated into the personal computer 33 but may be manufactured separately as an attachment to various computers.

As in the first embodiment, the signal strength of the communication signal is so set that the human body communication is not possible if two persons are involved as a communication medium.

The image detector unit 63 takes picture images of an operator seated on the operator's seat 12 and facing the display unit 35 and detects whether any operator is present in front of the display unit 35 and monitors whether any change of the operator arises. The seat sensor 70 transmits to the personal computer 33 the information indicating whether any operator is seated on the operator's seat as in the first embodiment.

The personal computer 33 includes a central processing unit (CPU) 53, which controls the log-in operation and the log-off operation based on the control signal inputted from the check unit 23, the information of the image detector unit 63 and the information of the seat sensor 70.

The electric configuration of the operation control system 3 is shown in FIG. 9 in more detail under an assumption that the operator is seated on the operator's seat 12 and taking the in-blood alcohol concentration measurement check. The transmission signal outputted from the transmitter circuit 10 is inputted to the operator as the communication signal through the transmitter interface 11.

The transmitter circuit 10 is configured to include its identification code (ID) varying from seat to seat, that is, from person to person, in the transmission signal.

The transmitter circuit 10 is further configured to operate at least when the log-in is requested after the electric power supply is turned on. For this purpose, the transmitter circuit 10 may be configured to receive a notification of a request of log-in by radio communication from the personal computer 33 or be persistently operable.

The check unit 23 includes a breath-air alcohol detector 213 in addition to the electromotive force detector 217, the receiver unit 40 and the receiver interface 220.

The receiver interface 220 is configured to be wound about a part (top end of a tubular part of the check unit 23), which the operator bites to blow the breath air into the check unit 23. Accordingly, when the operator tries to take the alcohol check, the mouth of the operator comes sufficiently close to the receiver interface 220 for establishment of human body communication. Thus, the communication signal applied to the operator is inputted to the receiver unit 40.

The breath-air alcohol detector 213 is configured to analyze the components included in the breath air and generate an electromotive force corresponding to the in-blood alcohol concentration. The electromotive force is detected by the electromotive force detector 217 as in the first and the second embodiments, the electromotive force detector 217 applies the check signal to the receiver unit 40. The receiver unit 40 inputs generates the control signal based on the reception signal and the check signal and applies it to the CPU 53 of the personal computer 33.

The transmitter side is grounded by legs of the operator and the receiver side is grounded by the grounding wire of the personal computer 33.

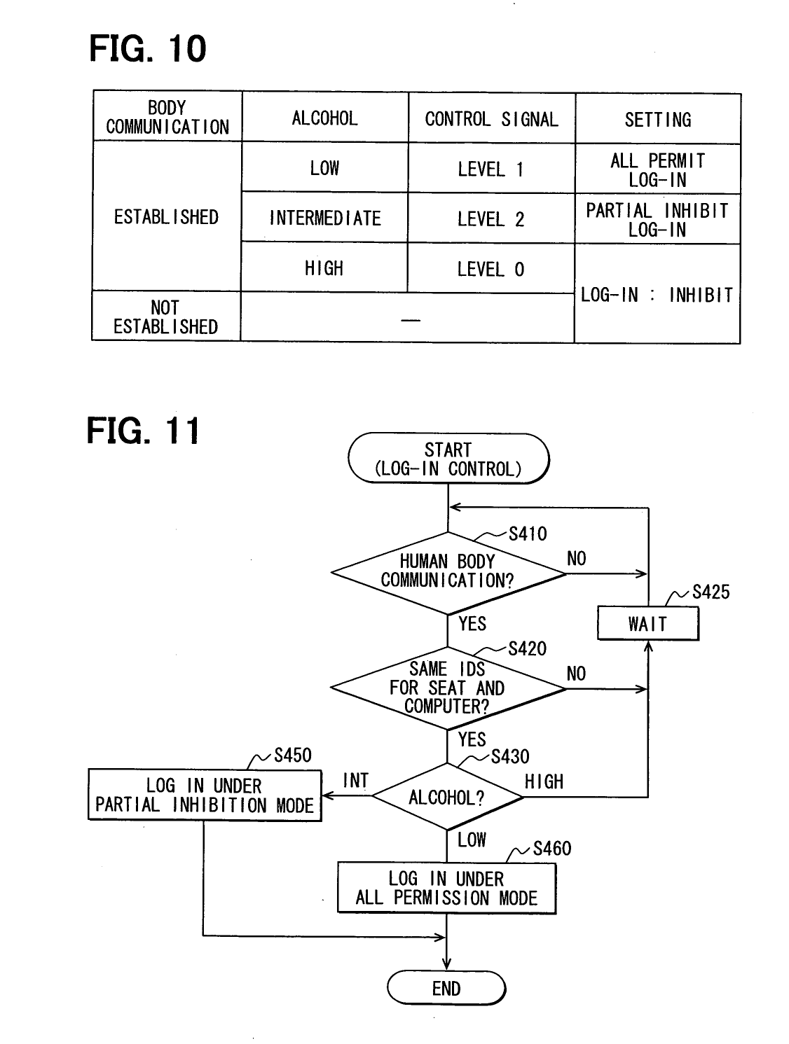

The CPU 53 performs log-in control based on the control signal inputted from the check unit 23. The principle of control is shown in FIG. 10. Specifically, the table of FIG. 10 shows the relation among the human body communication, the alcohol concentration, the control signal and the setting of the personal computer.

As defined in the table, if the human body communication is not established, no control signal is generated and the log-in is inhibited. Thus, the personal computer 33 is set to the inhibition state. If the in-blood alcohol concentration is high, that is, higher than a predetermined high threshold level, under the condition that the human body communication is established, the control signal takes a voltage level 0 and the log-in is inhibited.

If the in-blood alcohol concentration is intermediate, that is, lower than the predetermined high threshold level but higher than a predetermined low threshold level (lower than the predetermined high threshold level), under the condition that the human body communication is established, the control signal takes a voltage level 1 and the log-in is performed in a partial operation inhibition mode. “Partial operation” section operations such as “delete” operation, which will cause unwanted results, when operated erroneously. The partial operation inhibition mode is for inhibiting such operations. Thus the personal computer 33 is set to a partial inhibition state.

If the in-blood alcohol concentration is low, that is, lower than the predetermined low threshold level, under the condition that the human body communication is established, the control signal takes a voltage level 2 and the log-in is performed in an all operation permission mode. The all operation permission mode is a normal mode, in which no operation is inhibited. Thus, the personal computer 33 is set to a permission state.

The log-in control, routine is primarily performed by the CPU 53 as shown in FIG. 11 when the log-in is requested, that is, after the electric power supply is turned on, the personal computer 33 is re-started or logged off.

In the log-in control routine, it is first checked whether the human body communication is established (S410) by checking whether the control signal is received from the check unit 23. If it is determined that the human body communication is not established (NO at S410), S410 is repeated until the human body communication is established. If it is determined that the human communication is established (YES at S410), it is checked whether the seat ID included in the control signal is the same as the computer ID of the personal computer 33 (S420). If it is determined that the two IDs are not the same (NO at S420), the processing returns to S40 after waiting for a predetermined wait period (S425). This waiting is for inhibiting the log-in during the wait period.

If it is determined that the seat ID and the computer ID are the same (YES at S420), it is checked whether the in-blood alcohol concentration is high, intermediate or low (S430). If it is determined that the in-blood alcohol concentration is high, the processing returns to S410 after waiting for the time period (S425).

If it is determined that the in-blood alcohol concentration is intermediate, the personal computer 33 is logged in under the partial operation inhibition mode (S450). If it is determined that the in-blood alcohol concentration is low, the personal computer 33 is logged in under the all operation permission mode (S460).

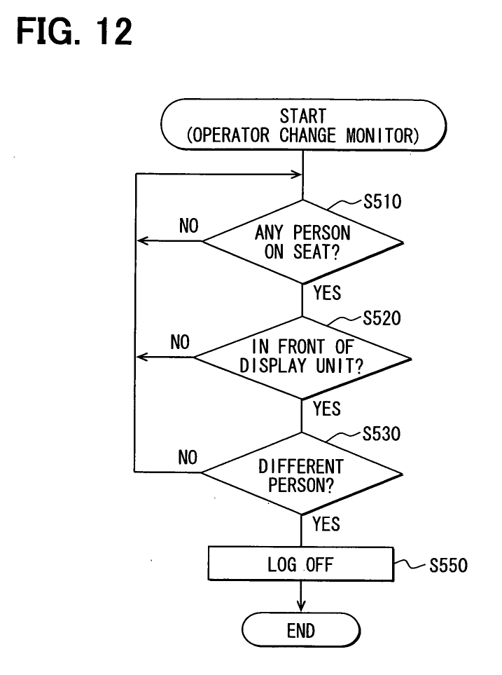

Any change of an operator is monitored by an operator change monitoring routine shown. in FIG. 12. This routine is also primarily performed by the CPU 53 when the personal computer 33 is logged in.

It is first checked whether any thing or person is on the operator's seat 12 based on the information produced by the seat sensor 70 (S510). If it is determined based on the information of the seat sensor 70 that a person is on the operator's seat 70 (YES at S510), it is further checked based on information produced by the image detector unit 63 whether the person is seated in front of the display unit 35 (S520). This check (S520) is executed because the operator's seat (chair) is movable. If the person moves away from the front surface of the display unit 35 while still being seated on the operator's seat 12, it is determined that no person is in front of the display unit 35.

If it is determined that the person is not in front of the display unit 35 (NO at S520), the processing returns to S510.

If it is determined that the person is in front of the display unit 35 (YES at S520), it is checked whether a different person is seated on the operator's seat 12 in front of the display unit 35 (S530) when the original person leaves the operator's seat 12 or moves away from the display unit 35 and somebody else comes to the operator's seat 12 and sits in front of the display unit 35. Specifically, this check may be made by storing the picture image of the original person based on the information of the image detector unit 63 and comparing a picture image of the next person seated on the operator's seat 12 with the stored image of the original person. If it is determined that the next person is the same as the original person and not the different person (NO at S530), the processing returns to S510 followed by S520.

If it is determined that the next person is different from the original person, that is, the operator is changed (YES at S530), the personal computer 33 is logged off (S550) and this routine is finished.

According to the third embodiment, the personal computer 33, which has been logged in, is protected from being operated by a drunk operator. Because the drunk operator is not permitted to perform the log-in operation, and the personal computer 33 is logged off when a different person tries to operate the personal computer 33 after the normal person, who is not drunk, has logged in.

In the similar manner as in the first embodiment, the signal strength of the communication signal is so set that the human body communication may not be established through two or more persons as a communication medium. Accordingly, it is prevented that the personal computer 33 is logged in by the other person, who holds a hand of the drunk operator sitting on the operator's seat 12.

In the third embodiment, it is possible to log off the personal computer 33 each time the operator, who has logged in, leaves the operator's seat 12. In this case, even if the same person returns to the operator's seat 12 in a moment, the personal computer 33 need be logged in again. However, it is possible to surely prevent that the personal computer 33, which has been logged in, is operated by a drunk person, even if the drunk person is inadvertently permitted by erroneous image recognition based on the picture image.

It is also possible to inhibit only a part of operation (partial operation) performed after the log-in without inhibiting the log-in based on the in-blood alcohol concentration. It is only the operator, who is seated on the specified operator's seat 12, that is permitted to log in. Therefore, any other persons, who are not on the operator's seat, are inhibited from logging in the personal computer 33.

It is also prevented that the personal computer 33 is logged in by a person, who is seated on a different seat other than the specified operator's seat 33, even if the different seat has a transmission circuit for performing human body communication. This is because the inhibition of log-in of each personal computer is released only when human body communication is established between the transmitter circuit 10 of the operator's seat 12 pre-specified to the particular personal computer 33.

It is possible to integrate the check unit 23 with the personal computer 33.

It is possible to perform communication between the check unit 23 and the personal computer 33 by not a USB connection but by a radio wave. In this instance, a communication technology (for example, JP 2009-154689), which does not form the human body communication in a closed circuit, may be employed.

### Fourth Embodiment

An operation control system 4 according to the fourth embodiment is provided for preventing a drunk person to operate a personal computer under the log-in condition as in the third embodiment. Specifically, when the measured in-blood alcohol concentration of an operator is higher than a predetermined threshold level, the personal computer is prevented from being logged in or it is forcibly logged-off.

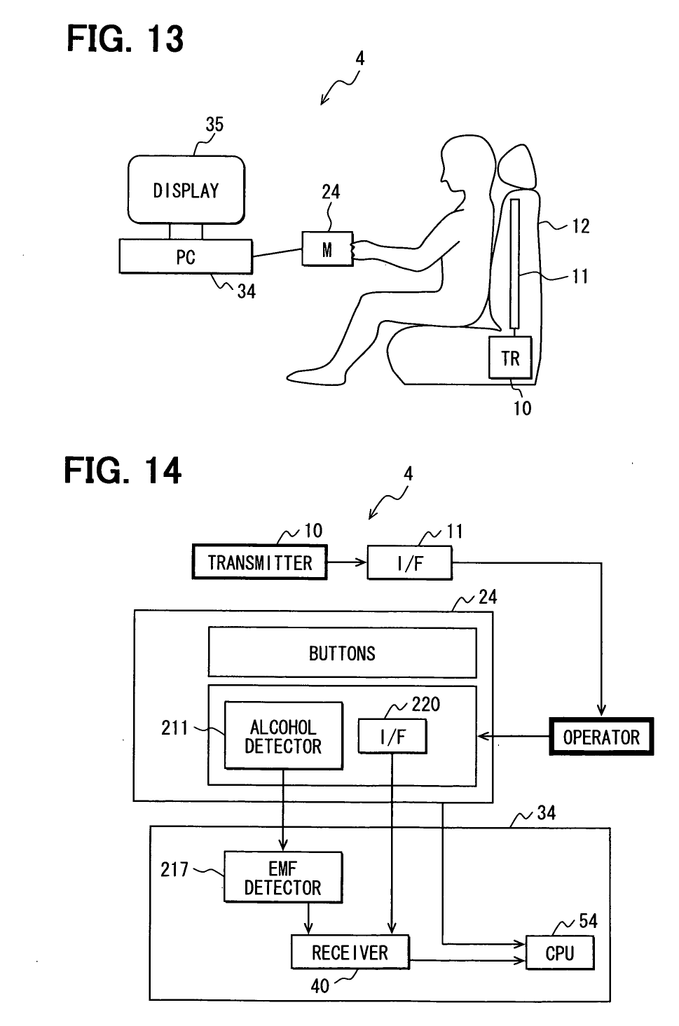

As shown in FIG. 13, the operation control system 4 includes a mouse 24, a personal computer 34 and a display unit 35, in addition to the transmitter circuit 10 and the transmitter interface 11, which are provided in the operator's seat 12.

The mouse 24 is provided to measure the in-blood alcohol concentration.

The personal computer 34 controls the log-in operation and the log-off operation based on the reception signal and the check signal inputted from the mouse 24. The reception signal and the control signal include identification code (ID) varying from seat to seat as in the third embodiment.

The signal strength of the communication signal is set to correspond to the human body communication through only one human body as in the foregoing embodiments.

The operation control system 4 is configured as shown in FIG. 14, in which the operator's hand is assumed to be in touch with the mouse 24. The mouse 24 includes the optical alcohol detector 211 and the receiver interface 220 thereby to measure the in-blood alcoholic concentration when an operator touches the mouse 24. When the operator's hand touches the mouse 24, it is sufficiently close to the receiver interface 220 so that the human body communication is established. The personal computer 34 includes a CPU 54 in addition to the receiver 40 and the electromotive force detector 217.

The CPU 54 is configured to control the log-in operation and the log-off operation based on the control signal of the receiver unit 40 as in the third embodiment. The transmitter circuit 10 is configured to be operative at least as long as the power is supplied to the personal computer 34. The transmitter circuit 10 therefore may be configured to be operative persistently or operative in response to a notification supplied from the personal computer 34 by a radio wave.

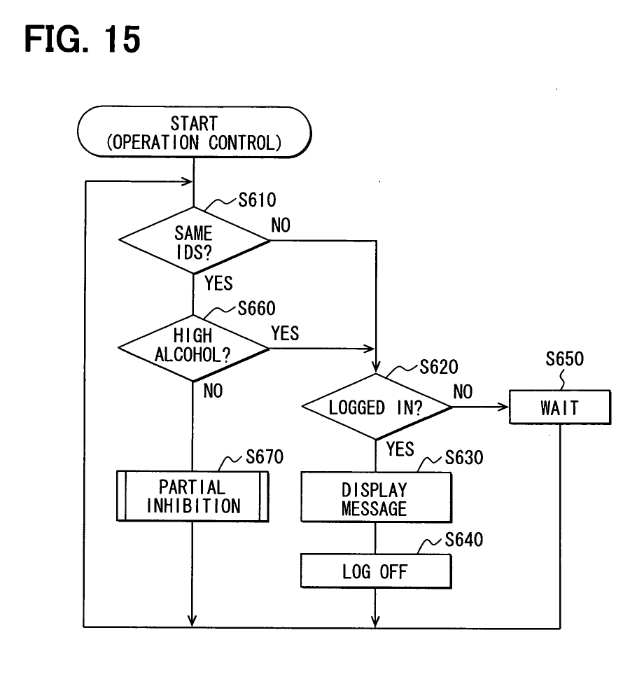

The personal computer 34 is configured to perform an operation control routine as shown in FIG. 15, which is primarily executed by the CPU 54. This routine is started when the human body communication is established and forcibly terminated when the human body communication is disrupted. The personal computer 34 is so configured that the log-in operation may be performed by a simple manipulation (for example, one click) on the mouse 24.

It is first checked whether the seat ID of the operator's seat 12 included in the control signal and the ID of the personal computer 34 are the same (S610). If it is determined that the two IDs are not the same (NO at S610), it is further checked whether the personal computer 34 has already been logged in (S620). If it is determined that the personal computer 34 has been logged in (YES at S620), a message of “The personal computer will be logged off in thirty seconds.” is displayed on the display unit 35 (S630). The personal computer 34 is logged off after waiting for thirty seconds (S640).

If it is determined that the personal computer 34 is logged off and not logged in (NO at S620), the processing returns to S610 after waiting for a predetermined time period (S650). During this time period, the log-in operation is inhibited.

If it is determined that the compared two IDs are the same (YES at S610), it is further checked whether the in-blood alcohol concentration is high, that is, the measurement alcohol concentration is higher than a predetermined high threshold level (S660). If it is determined that the alcohol concentration is high (YES at S660), S620 and subsequent steps S630, S640 and S650 are executed as described above. If it is determined that the alcohol concentration is not high (NO at S660), a partial inhibition process is performed (S670) and the processing returns to S610.

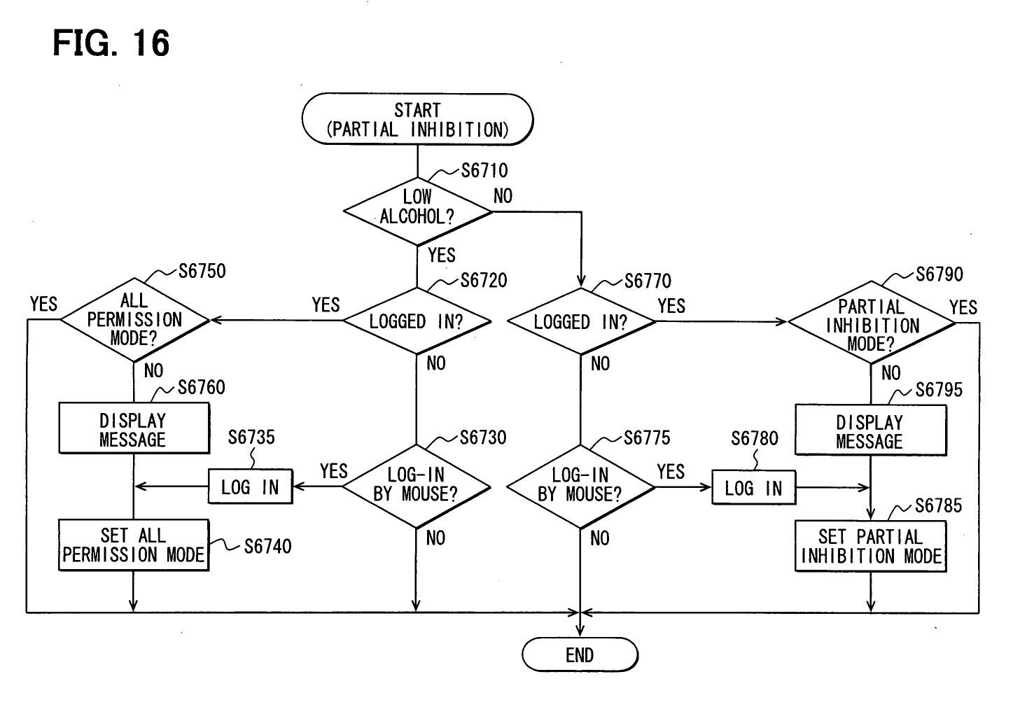

The partial inhibition process S670 is executed as shown in FIG. 16. It is first checked whether the in-blood alcohol concentration is low, that is, the measured alcohol concentration is lower than a predetermined low threshold level (S6710). If the in-blood alcohol concentration is low (YES at S6710), it is further checked whether the personal computer 34 is logged in (S6720). If it is determined that the personal computer 34 is logged off (NO at S6720), it is further checked whether the mouse 24 is operated to log in the personal computer 34 (S6730). If it is determined that the mouse 24 is not operated (NO at S6730), the partial inhibition process is finished. In this case, the processing returns to S610.

If it is determined that the log-in operation is made by the mouse 24 (YES at 6730), the personal computer 34 is logged in (S6735) and the personal computer 34 is set to the all permission mode (S6740) described in the third embodiment. The processing returns to S610.

If it is determined that the personal computer 34 is logged in (YES at S6720), it is further checked whether the personal computer 34 is set in the all permission mode (S6750). If it is determined that the personal computer 34 is set to the all permission mode (YES at S6750), this routine is finished and the processing returns to S610. If it is determined that the personal, computer 34 is not set in the all permission mode (NO at S6750), a message of “The personal computer will proceed to the all permission mode.” is displayed on the display unit 35 (S6760). The personal computer 34 is set to the all permission mode (S6740), thus finishing this routine and returning to S610 as described above.

If it is determined that the in-blood alcohol concentration is higher than the predetermined low threshold level (NO at S6710), it is further checked whether the personal computer 34 is logged in (S6770). This alcohol level corresponds to the intermediate level. If it is determined that the personal computer 34 is logged off (NO at S6770), it is further checked whether the mouse 24 is operated to log in the personal computer 34 (S6775). If it is determined that the mouse 24 is not operated (NO at S6775), the partial inhibition process is finished. In this case, the processing returns to S610.

If it is determined that the log-in operation is made by the mouse 24 (YES at 6775), the personal computer 34 is logged in (S6780) and the personal computer 34 is set to the partial inhibition mode (S6785) described in the third embodiment. The processing returns to S610.

If it is determined that the personal computer 34 is logged in (YES at S6770), it is further checked whether the personal computer 34 is set in the partial inhibition mode (S6790). If it is determined that the personal computer 34 is set to the partial inhibition mode (YES at S6790), this routine is finished and the processing returns to S610.

If it is determined that the personal computer 34 is not set in the partial inhibition mode (NO at S6790), a message of “The personal computer will proceed to the partial inhibition mode.” is displayed on the display unit 35 (S6795). The personal computer 34 is set to the partial inhibition mode (S6785), thus finishing this routine and returning to S610 as described above.

According to the operation control system 4, the personal computer 34, which is logged in, is protected from being operated by a drunk operator. Even when an operator starts drinking alcohol after logging in the personal computer 34, the personal computer 34 is logged off by detecting the drinking. Since the change of operators need not be monitored, the hardware and software of the operation regulation system 4 is simplified.

By way of the human body communication, the alcohol measurement result is specific to the operator and not anybody else. Therefore, it is prevented that the log-in and log-off are controlled based on. the alcohol measurement result of other persons. It is possible that the operation of the personal computer 34 after having been logged in can be inhibited partly in accordance with the degree of in-blood alcohol concentration.

The CPU 54 may be programmed to accept the operation performed through the mouse 24 only when the human body communication is maintained. In this instance, the operator need be seated on the operator's seat 12. As a result, the personal computer 34 is protected form being operated erroneously by a nearby person other than the operator.

The present invention is not limited to the disclosed embodiments but may be modified in many other ways as exemplified below. In the foregoing first to fourth embodiments, the transmission and reception of signals may be reversed. For example, in the first embodiment, the transmitter circuit 10 and the receiver unit 40 may be arranged so that the communication signal is transmitted from the engine start button switch 21 and received inside the driver's seat 12.

In the third and fourth embodiments, seat ID may be transmitted by a radio wave in place of the human body communication. The transmitter circuit 10 may be grounded to the grounding part of the operator's seat 12. The transmitter interface 11 and the receiver interface 220 may be electrodes. The human body communication may be made by using electric field or electromagnetic filed in place of magnetic field.

What is claimed is:

1. An operation control system for controlling operation of a control subject, the operation control system comprising:
setting section configured to set the control subject to an inhibition state or a permission state based on a body condition of an operator seated on a seat for operating the control subject, so that the operation of the control subject is limited in the inhibition state and permitted in the permission state;
communication section including a transmitter and a receiver and configured to perform communication through a human body of the operator, which is present closely to the transmitter and the receiver; and
condition checking section including a detector and configured to check the body condition of the operator under a condition that the detector is positioned closely to the operator;
wherein one of the transmitter and the receiver is provided at the seat and the other of the transmitter and the receiver is provided at the detector of the condition checking section, and
the setting section is configured to set the inhibition state or the permission state based on a check result of the condition checking section outputted in a condition that the human body communication is established.

2. The operation control system according to claim 1, wherein:
the setting section sets the control subject to the inhibition state with respect to a start operation of the control subject, when the human body communication is not established by the communication section or the check result of the condition checking section indicates improper body condition of the operator; and
the setting section sets the control subject to the permission state with respect to the start operation of the control subject, when the human body communication is established by the communication section and the check result of the condition checking section indicates proper body condition of the operator.

3. The operation control system according to claim 2, wherein:
the control subject is a vehicle;
the seat is a driver's seat in the vehicle;
the condition checking section checks alcohol concentration of the operator seated on the seat as the body condition;
the setting section sets the vehicle to the inhibition state thereby to inhibit an operation for enabling travel of the vehicle, when the human body communication is not established by the communication section or the check result of the condition checking section indicates that the operator is drunk; and
the setting section sets the vehicle to the permission state thereby to permit the operation for enabling travel of the vehicle, when the human body communication is established by the communication section and the check result of the condition checking section indicates that the operator is not drunk.

4. The operation control system according to claim 3, wherein:
the condition checking section checks alcohol concentration included in a breath of the operator seated on the seat; and
the transmitter or the receiver is provided at a position to allow the operator to bite by a mouth while being seated on the seat.

5. The operation control system according to claim 3, further comprising:
seating checking section configured to detect the operator seated on the driver's seat; and
the setting section sets the vehicle to the inhibition state to inhibit the travel of the vehicle, when the seating checking section detects no operator on the driver's seat.

6. The operation control system according to claim 3, further comprising:
driver change detecting section configured to detect a change of a driver seated on the driver's seat; and
the setting section sets the vehicle to the inhibition state to inhibit the travel of the vehicle, when the driver change detecting section detects the change of a driver under a condition that the vehicle is set to the permission state.

7. The operation control system according to claim 2, wherein:
the control subject is a computer;
the condition checking section checks alcohol concentration of the operator seated on the seat as the body condition;
the setting section sets the computer to the inhibition state thereby to inhibit an switching operation of the computer from a log-off state to a log-in state, when the human body communication is not established by the communication section or the check result of the condition checking section indicates that the operator is drunk; and
the setting section sets the computer to the permission state thereby to permit the log-in of the computer, when the human body communication is established by the communication section and the check result of the condition checking section indicates that the operator is not drunk.

8. The operation control system according to claim 7, further comprising:
seating checking section configured to check, whether the operator is seated on the seat and the operator is present in front of a display unit of the computer,
wherein the setting section sets the inhibition state thereby to log off the computer, when the seating checking section indicates that the operator is not seated on the seat or the operator is not present in front of the display unit under a condition that the computer is logged in.

9. The operation control system according to claim 7, further comprising:
seating checking section configured to check whether the operator is seated on the seat and the operator is present, in front of a display unit of the computer; and
operator change detecting section configured to detect a change of an operator seated on the seat after the seating checking section determines that the operator is not seated on the seat or the operator is not present in front of the display unit,
wherein the setting section sets the inhibition state thereby to log off the computer, when the operator change detecting section detects the change of an operator under a condition that the computer is logged in.

10. The operation control system according to claim 1, wherein:
the detector is provided in an operation device of the control subject so that the body condition is detected by the detector when the operation device is touched by the operator to operate the control subject; and
the setting section sets the control subject to the inhibition state, when a check result of the condition checking section indicates an improper body condition under a condition that the human body communication is established by the communication section.

11. The operation control system according to claim 10, wherein:
the control subject is a vehicle;
the seat is a driver's seat in the vehicle;
the detector is provided on a steering wheel of the vehicle;
the condition checking section checks alcohol concentration in blood of the operator by checking a hand of the operator touching the steering wheel; and
the setting section sets the vehicle to the inhibition state thereby to inhibit an operation for enabling travel of the vehicle if the vehicle is not permitted to travel, and thereby to inhibit the travel of the vehicle if the vehicle is permitted to travel, when the human body communication is established by the communication section and the check result of the condition checking section indicates that the operator is drunk.

12. The operation control system according to claim 10, wherein:
the control subject is a computer;
the condition checking section checks alcohol concentration of the operator seated on the seat as the body condition by checking a hand of the operator touching the operation device; and
the setting section sets the computer to the inhibition state thereby to inhibit an switching operation of the computer from a log-off state to a log-in state if the computer is logged off, and to log off the computer if the computer is logged in, when the human body communication is established by the communication section and the check result of the condition checking section indicates that the operator is drunk.

13. The operation control system according to claim 5, wherein:
the setting section sets the vehicle to the inhibition state thereby to inhibit the travel of the vehicle only when the vehicle is at rest.

14. The operation control system according to claim 3, wherein:
the operation to enable the travel of the vehicle is a starting operation of an engine of the vehicle.

15. The operation control system according to claim 5, wherein:
the travel of the vehicle is inhibited by a forced stop of an engine of the vehicle.

16. The operation control system according to claim 7, wherein:
the setting section sets the computer to a partial inhibition state thereby to inhibit only a part of operation of the computer, when the check result of the condition checking section indicates that the alcohol concentration of the operator is between a predetermined low threshold level and a predetermined high threshold level, over which the operator is determined to be drunk.

17. The operation control system according to claim 1, wherein:
the transmitter is provided close to the human body seated on the seat and transmits a signal including a specific identification code; and
the setting section determines that the body communication is established by the communication section, only when the specific identification code included in the signal received by the receiver is the same as an identification code provided in the setting section.

18. The operation control system according to claim 1, wherein:
the transmitter is configured to transmit a signal having a strength, with which the human body communication is established by only one operator.

19. An operation control method for controlling operation of a control subject, the operation control method comprising:
transmitting a communication signal from a transmitter provided in a seat provided for the operator to operate the control subject by touching a part of the control subject;
checking whether the communication signal is received by a receiver provided in the part of the control subject through a body of the operator seated on the seat;
checking an in-blood alcohol concentration of the operator by a detector provided in the part of the control subject under a condition that the part of the control subject is touched by the operator seated on the seat; and
permitting the control subject to be operated by the operator only when check results of the checking steps indicate that the communication signal is received by the receiver in the part of the control subject and the in-blood alcohol concentration is lower than a predetermined threshold level.

20. The operation control method according to claim 19, wherein:
the communication signal is transmitted from a driver's seat in a vehicle;
the in-blood alcohol concentration is detected by the detector provided in one of an engine start switch and a steering wheel of the vehicle; and
an engine of the vehicle is permitted to be started in response to an operation of the engine start switch, only when the communication signal is received by the receiver and the in-blood alcohol concentration is lower than the predetermined threshold level.
